(19) United States
(12) Patent Application Publication
(10) Pub. No.: US 2018/0221607 A1
(43) Pub. Date: Aug. 9, 2018

(54) CARDIAC, CARDIOPULMONARY, AND/OR HEMODYNAMIC PHENOTYPING

(71) Applicant: KONINKLIJKE PHILIPS N.V., EINDHOVEN (NL)

(72) Inventors: JOACHIM KAHLERT, AACHEN (DE); BART KROON, EINDHOVEN (NL)

(21) Appl. No.: 15/750,530

(22) PCT Filed: Aug. 4, 2016
(86) PCT No.: PCT/EP2016/068706

§ 371 Date: Feb. 6, 2018

(30) Foreign Application Priority Data
Aug. 7, 2015 (EP) 15180220.4

(51) Int. Cl. A61M 16/00

(52) U.S. Cl. CPC A61M 16/024; A61M 2230/30; A61M 16/101; A61M 2230/20; A61M 2205/3303

(57) ABSTRACT

Various systems, apparatus and methods are described herein for generating cardiac, cardiopulmonary, and/or hemodynamics phenotypes of patients for use in various applications. In various embodiments, a ventilation support apparatus connected to a patient may be operated to perform a therapeutic maneuver selected to cause one or more targeted receptors of the patient to initiate an autoregulation response in the patient. In various embodiments, a measurement may be taken of one or more changes in a vital sign of the patient such as blood pressure that is caused by initiation of the autoregulation response in the patient. In various embodiments, a cardiac, cardiopulmonary, or hemodynamic phenotype of the patient may be generated based on the measured one or more changes.

### TECHNICAL FIELD OF THE INVENTION

The present invention is directed generally to healthcare. More particularly, various inventive methods and apparatus disclosed herein relate to cardiac, cardiopulmonary, and/or hemodynamic phenotyping to provide better health care.

### BACKGROUND OF THE INVENTION

Blood pressure is one of the most commonly-measured vital signs in human patients. It may be measured to learn about the cardiac and/or hemodynamic status of the patient, as well about sympathetic/parasympathetic activity of the patient. Measuring blood pressure is a relatively easy, minimally invasive and/or non-invasive procedure to determine whether a patient is within a normative range or deviates from that range. Blood pressure is not constant. It changes due to a variety of factors, such as physical activity, physical and emotional stress, environmental factors, physiological alterations, intermittent and/or chronic diseases or disorders, and so forth.

Ventilation support systems such as continuous positive airway pressure (“CPAP”) machines and or bi-level positive airway pressure (“BiPAP”) are designed to, among other things, rebalance a patient's inspiratory and expiratory airflow. By increasing tidal volume and minute volume, sufficient oxygen may be delivered into the patient's lungs. However, operation of ventilation support systems using improper ventilation parameters may negatively impact the cardiac system. For instance, an increased intrathoracic pressure (“ITP”) may be caused by elevated positive end expiratory pressure (“PEEP”). An elevated PEEP may hamper the venous return flow of blood into the right atrium of the patient's heart. Moreover, increased ITP may result in a higher afterload on the right ventricle of the patient's heart, which may impede blood flow into the patient's lungs. Under such compromised ventilation conditions, the patient's blood pressure may increase as part of an autoregulation response initiated by the central nervous system to sufficiently pump blood. Persistent elevated blood pressure may lead to a structural remodeling of the patient's heart and lungs, and is a leading cause of chronic hypertension.

In hypertensive patients, a vasodilator drug therapy may be prescribed to lower the patient's blood pressure. However, this treatment does not treat the underlying cause of the patient's hypertension; it is purely for relief of a symptom (high blood pressure). While such medication therapy may stop or slow down secondary disease exacerbation, it does not attempt to take advantage of the body's own mechanisms for regulating blood pressure, such as autoregulation initiated by the central nervous system in response to signals received from chemoreceptors and various types of mechanoreceptors, such as baroreceptors. Thus, there is a need in the art to learn about a patient's autoregulation response to triggering of various receptors in order to better treat underlying causes of hypertension and/or ventilation/perfusion imbalance.

US 2014/0202455 discloses an apparatus for providing close-loop control of the operation of a ventilator device based on physiological parameters.

### SUMMARY OF THE INVENTION

The present disclosure is directed to inventive methods and apparatus for determining cardiac, cardiopulmonary, and/or hemodynamic phenotypes to improve healthcare. For example, a ventilation support system such as a CPAP may be operated to perform one or more therapeutic maneuvers selected to cause a patient's chemoreceptors or baroreceptors to trigger a central nervous system autoregulation response. Autoregulation responses may include but are not limited to cardiac and/or hemodynamic responses. Then, a change in the patient's vital signs (e.g., blood pressure) that results from the autoregulation response may be measured to determine a patient's cardiac, cardiopulmonary, and/or hemodynamic phenotype. This phenotype may be used for a variety of purposes, such as clustering the patient with other similar patients in healthcare studies, selectively operating the patient's ventilation support system to more effectively treat the patient in accordance with their personalized phenotype, and so forth. In this manner, an increase of a patient's blood pressure and/or manifestation of chronic hypertension in the patient may be prevented by leveraging the patient's own central nervous system autoregulation responses.

Generally, in one aspect, a ventilation support system may include a ventilation support apparatus and a controller. In various embodiments, the controller may be configured to: cause the ventilation support apparatus to perform a therapeutic maneuver selected to cause a targeted receptor to initiate an autoregulation response in a patient connected to the ventilation support system; measure one or more changes in one or more vital signs caused by initiation of the autoregulation response in the patient; generate a cardiac, cardiopulmonary, or hemodynamic phenotype of the patient based on the measured one or more changes; and operate the ventilation support apparatus in accordance with the phenotype.

In various embodiments, the one or more vital signs may include blood pressure. In various versions, the controller may be further configured to: operate the ventilation support apparatus to alter an applied treatment parameter in a series of discrete steps; and measure one or more changes in the blood pressure caused by each of the series of discrete steps. In various versions, the controller may be further configured to operate the ventilation support apparatus to incrementally increase or decrease a continuous positive airway pressure. In various versions, the controller may be further configured to operate the ventilation support apparatus to activate or deactivate positive or negative airway pressure. In various versions, the controller may be further configured to operate the ventilation support apparatus to incrementally increase or decrease concentration of one or more components of air supplied to the patient.

In various embodiments, the controller may be further configured to: provide output prompting medical personnel to reposition the patient between a plurality of bodily positions in a predetermined manner; and measure one or more changes in blood pressure, or receive one or more indications of one or more changes in blood pressure, caused by the repositioning of the patient. In various embodiments, the targeted receptor may be chemoreceptors. Operating the ventilation support system to perform the therapeutic maneuver may include operating the ventilation support system to alter a chemical composition of blood of the patient. In various versions, the targeted receptor may be baroreceptors. Operating the ventilation support system to perform the therapeutic maneuver may include operating the ventilation support system to alter a vessel or airway pressure of the patient.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 schematically illustrates an example ventilation support system configured with selected aspects of the present disclosure, in accordance with various embodiments.

FIG. 2 depicts an example method for performing cardiac, cardiopulmonary, and/or hemodynamic phenotyping, in accordance with various embodiments.

### DETAILED DESCRIPTION OF THE EMBODIMENTS

Blood pressure is one of the most commonly-measured vital signs in human patients, and may be used to learn about the cardiac and/or hemodynamic status of the patient, as well about sympathetic/parasympathetic activity of the patient. Blood pressure changes due to a variety of factors, such as physical activity, physical and emotional stress, environmental factors, physiological alterations, intermittent and/or chronic diseases or disorders, and so forth. One such factor is use of a ventilation support system such as a CPAP and/or BiPAP device. Operation of such ventilation support systems using improper ventilation parameters may negatively impact the cardiac system and lead to elevated blood pressure, which if persistent may cause chronic hypertension. Moreover, while a vasodilator drug therapy may be prescribed to lower a patient's blood pressure, it does not treat the underlying cause of the patient's hypertension. Thus, there is a need in the art to learn about a patient's autoregulation response to triggering of various receptors, so that medical treatment may be tailored to rebalance the patient's blood pressure using the body's own mechanisms for regulating blood pressure, such as chemo- and baroreceptor initiated autoregulation.

In view of the foregoing, various embodiments and implementations of the present invention are directed to determining a patient's cardiac, cardiopulmonary, and/or hemodynamic phenotype, and using the phenotype to provide improved health care. In various embodiments, disclosed techniques may be used to perform diagnostic and therapy planning using ventilation support systems and apparatus. One technical advantage of these techniques is that a patient's blood pressure may be lowered by balancing the ventilation and perfusion in the patient's lung, rather than by simply using vasodilator drug therapies. In some embodiments, this balancing may be achieved by deliberately triggering one or more autoregulation responses of the patient's central nervous system.

For example, a persistent and/or intermittent raise of systemic and pulmonary blood pressure in a patient is often the response to a cardiopulmonary ventilation/perfusion mismatch. This ventilation/perfusion mismatch may be caused either by insufficient ventilation (which may be caused by pulmonary diseases, sleep disordered breathing) or by insufficient perfusion of the patient's lung (which may be caused by cardiovascular diseases, increased intrathoracic pressure, or heart failures). Techniques described herein may be employed to cause targeted chemoreceptors and baroreceptors to initiate various autoregulation responses. Resulting changes in vital signs, such as blood pressure, heart rate, and stroke volume, may be measured to determine how best to treat the underlying causes of the cardiopulmonary ventilation/perfusion mismatch.

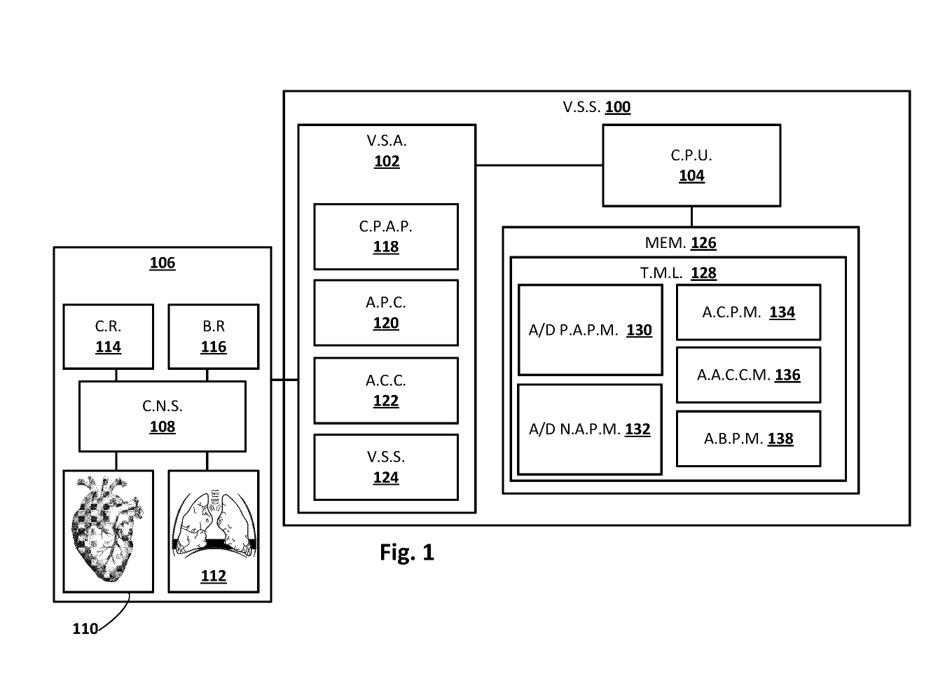

Referring to FIG. 1, in one embodiment, a ventilation support system 100 (“V.S.S.” in FIG. 1) may include a ventilation support apparatus 102 (“V.S.A.” in FIG. 1) communicatively coupled with a controller 104 (“C.P.U.” in FIG. 1”). In various embodiments, ventilation support apparatus 102 may take various forms, such as a CPAP device, a BiPAP device, or any other device that seeks to alter a manner in which a patient breathes. Various communication technologies may be used to communicatively couple ventilation support apparatus 102 with controller 104, including but not limited to one or more buses, one or more wired or wireless communication technologies (e.g., Wi-Fi, Bluetooth, etc.), and so forth.

Ventilation support apparatus 102 may be connected, e.g., by one or more medical personnel (not depicted, e.g., doctors, nurses), to a patient 106 during various procedures, such as a sleep study, using various mechanisms, such as a nasal mask, a nasal-oral mask, an oral mask, and so forth. Patient 106 includes a central nervous system 108 (“C.N.S.” in FIG. 1, includes various undepicted organs such as a brain, nerves, and a spinal cord), a heart 110, and one or more lungs 112, among other standard organs and body parts. Patient 106 may also include one or more receptors that may be targeted by various therapeutic maneuvers (described in more detail below) to initiate an autoregulation response in central nervous system 108.

For example, chemoreceptors 114 (“C.R.” in FIG. 1) such as carotid or aortic bodies may sense a chemical composition of blood of patient 106, and may relay that information to central nervous system 108. If the sensed chemical composition meets one or more criteria, such as blood oxygen or carbon dioxide being too high or low, central nervous system 108 may initiate various autoregulation responses, such as causing lungs 112 to expand or retract to a greater degree. Baroreceptors 116 (“B.R.” in FIG. 1) may be located within blood vessels (not depicted) of patient 106. Baroreceptor 116 activity may reflect changes in airway and vessel pressure, and/or cardiac and pulmonary lung tissue stretching. Baroreceptors 116 may relay information they sense to central nervous system 108. If the sensed pressure(s) and/or stretching meets one or more criteria, central nervous system 108 may initiate various autoregulation responses, e.g., to raise or lower blood pressure.

Ventilation support apparatus 102 may include various controls 118-122 that are operable, e.g., manually or by controller 104, to perform a variety of therapeutic maneuvers selected to cause a targeted receptor (e.g., 114 or 116) to initiate an autoregulation response by central nervous system 108. In particular, in some embodiments, one or more controls 118-122 may be operated to alter a treatment parameter applied to patient 106. Such alterations may occur sporadically, in a series of discrete steps, simultaneously, or in a variety of other sequences. In embodiments in which the alterations occur as a series or sequence of discrete steps, a vital sign sensor 124 (“V.S.S.” in FIG. 1) may be configured to measure a change in one or more vital signs (e.g., blood pressure, heart rate, stroke volume, etc.) of patient 106 caused by the discrete alteration. Controls 118-122 may be implemented with any combination of hardware and/or software. In some embodiments, controls 118-122 may include physical knobs, dials, sliders, buttons, and so forth, or rendered graphical elements on a graphical user interface, that a user may manipulate manually. Additionally or alternatively, one or more of controls 118-112 may be associated with one or more application programming interfaces (“API”) that are accessible by controller 104, e.g., so that controller 104 may issue commands to controls 118-122, e.g., during implementation of various therapeutic maneuvers.

For example, CPAP pressure control 118 (“C.P.A.P.” in FIG. 1) may be operable, e.g., by controller 104, to incrementally increase or decrease a continuous positive airway pressure (i.e., CPAP) applied to patient. Airway pressure control 120 (“A.P.C.” in FIG. 1) may be operable, e.g., by controller 104, to activate and/or deactivate positive and/or negative airway pressure (e.g., intrathoracic pressure) in patient 106. Air composition control 122 (“A.C.C.” in FIG. 1) may include an oxygen concentrator (not depicted) and/or a nitrogen enrichment device, and may be operable to incrementally increase or decrease levels of various constituent components of air supplied to patient 106, such as oxygen and/or nitrogen.

Controller 104 may be operably coupled with memory 126 (“MEM.” in FIG. 1). Memory 126 may come in various forms, such as read only memory (“ROM”), random access memory (“RAM”), flash memory, solid state memory, one or more hard drives, and so forth. In various embodiments, memory 126 may store a library 128 (“T.M.L.” in FIG. 1) of therapeutic maneuvers 130-138. Controller 104 may implement one or more maneuvers of library 128 to operate various controls (e.g., 118-122) of ventilation support apparatus 102 in a manner that targets receptors (e.g., 114, 116) to initiate autoregulation responses in central nervous system 108.

For example, controller 104 may implement one or more instructions comprising an activate/deactivate positive airway pressure maneuver 130 (“A/D P.A.P.M.” in FIG. 1) in order to cause controller 104 to operate airway pressure control 120 to (e.g., repeatedly) activate and/or deactivate positive airway pressure applied to patient 106. Similarly, controller 104 may implement one or more instructions comprising an activate/deactivate negative airway pressure maneuver 132 (A/D N.A.P.M.” in FIG. 1) in order to cause controller 104 to operate airway pressure control 120 to (e.g., repeatedly) activate and/or deactivate negative airway pressure applied to patient 106. In some embodiments, positive airway pressure maneuver 130 and/or negative airway pressure maneuver 132 may include instructions that cause controller 104 to operate airway pressure control 120 to rapidly change positive or negative airway pressure of patient 106 by various amounts, e.g., by five or ten cmH₂O. Vital sign sensor 124 may measure a change in blood pressure or other vital sign that occurs at each pressure level and may relay that information to controller 104.

Controller 104 may implement one or more instructions comprising an alter CPAP pressure maneuver 134 (“A.C.P.M.” in FIG. 1) to operate CPAP pressure control 118 to increase or decrease CPAP in discrete steps, e.g., by one centimeter of water (or “cmH₂O”), or by other amounts of pressure. This incrementing of CPAP may be set to occur periodically, for example every five seconds, every ten seconds, every fifteen seconds, every twenty seconds, every thirty seconds, every minute, every two minutes, and so forth. Vital sign sensor 124 may measure a change in blood pressure or other vital sign that occurs at each increment and may relay that information to controller 104.

Controller 104 may implement one or more instructions comprising an alter oxygen levels maneuver 136 (“A.A.C.C.M.” in FIG. 1) to operate air composition control 122 to increase or decrease oxygen in air breathed by patient 106 by various degrees. For example, in some embodiments, oxygen may be increased or decreased in steps of 5%. In some such embodiments, controller 104 may operate air composition control 122 to begin oxygen levels at 5%, and to increase in steps of 5% until 40% oxygen is reached. Vital sign sensor 124 may sense a change in blood pressure or other vital sign caused by each step of oxygen level change, and may relay this information to controller 104.

In some embodiments, controller 104 may implement one or more instructions of an alter bodily position maneuver 138 (“A.B.P.B.” in FIG. 1) to instruct medical personnel to alter a bodily position of patient 106. For example, controller 104 may provide output, e.g., on a display (not depicted), that instructs a nurse to move patient 106 between a lateral position and supine position. Vital sign sensor 124 may sense a change in blood pressure or other vital sign of patient 106 caused by each change in bodily position, and may relay this information to controller 104. As another example, controller 104 may provide output that instructs a nurse to reposition a protrusion of a tongue or mandible of patient 106. At each position of the protrusion of the tongue or mandible, vital sign sensor 124 may sense a change in blood pressure or other vital sign of patient 106, and may relay this information to controller 104.

In some embodiments, in addition to or instead of providing output to instruct medical personnel to reposition some bodily aspect of patient 106, controller 104 may automatically operate one or more components of ventilation support system 100, such as a tongue or mandible advancement device (not depicted), to reposition a bodily aspect of patient 106. Additionally or alternatively, controller 104 may be communicatively coupled to controls of a bed (not depicted) in which patient 106 sleeps, and controller 104 may cause the bed to reposition patient 106 between various positions.

In various embodiments, controller 104 may take the information relayed to it by vital sign sensor 124 in response to operation of the various controls 118-122, and may determine a cardiac, cardiopulmonary, and/or hemodynamic phenotype of patient 106. This phenotype may be used, e.g., by medical personnel, to provide treatment to patient 106 that better targets underlying causes of various ailments, such as inspiratory/expiratory airflow imbalance and/or chronic hypertension. For example, medical personnel may operate ventilation support system 100 in a manner that is tailored towards the phenotype of patient 106, and in particular, in a manner that aims to leverage the patient's own autoregulation responses to treat a disorder, rather than simply decreasing symptoms such as blood pressure or snoring.

In some embodiments, controller 104 may analyze the data it receives from vital sign sensor 124 to determine thresholds for various autoregulation responses, sensitivity of autoregulation responses, and/or limits (e.g., saturation) of autoregulation responses. In some embodiments, controller 104 may implement multiple different maneuvers at once or in sequence in order to operate ventilation support apparatus 102 in ways that trigger multiple different receptors (e.g., 114 and 116). Triggering multiple different receptors at once or in sequence may cause central nervous system 108 of patient 106 to initiate multiple different autoregulation responses. Controller 104 may analyze resulting changes in vital signs (e.g., blood pressure) sensed by vital sign sensor 124 to determine interdependences between the various autoregulation responses.

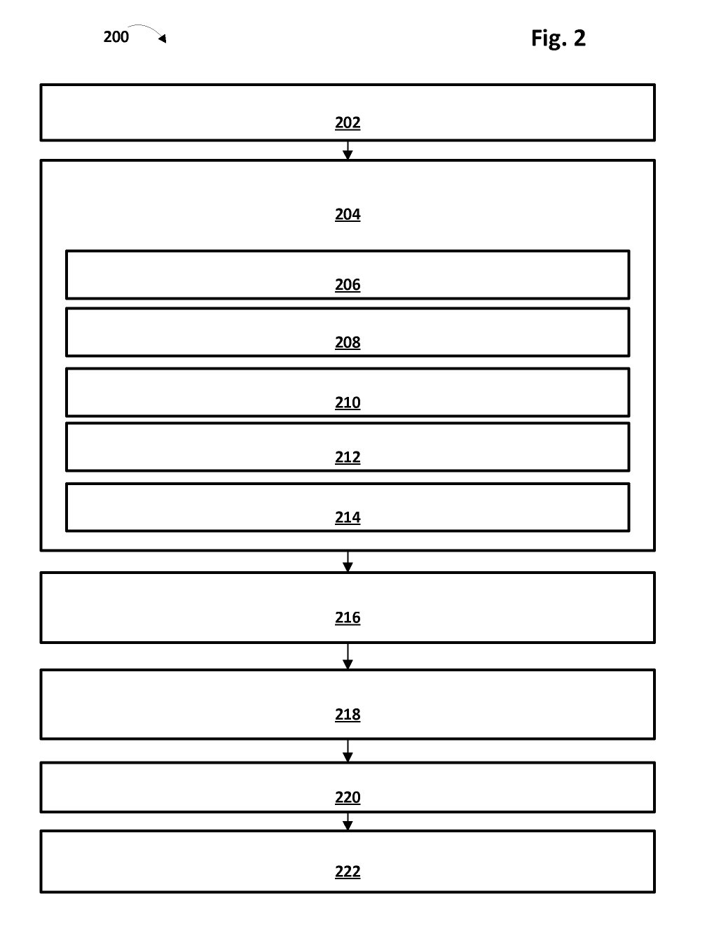

Referring now to FIG. 2, a method 200 of determining a patient's cardiac, cardiopulmonary, and/or hemodynamic phenotype, and for using this phenotype to provide improved treatment to the patient and/or to other patients that exhibit similar phenotypes is depicted. For convenience, the operations of flow charts are described with reference to a system that performs the operations. This system may include various components of ventilation support system 100. Moreover, while operations of method 200 are shown in a particular order, this is not meant to be limiting. One or more operations may be reordered, omitted or added.

At block 202, the ventilation support system (e.g., 100) may be connected to a patient (e.g., 106), e.g., by medical personnel such as a doctor or nurse. As noted above, various mechanisms may be used to connect the ventilation support system to the patient. For example, a nose mask may be positioned to cover the patient's nose, a nasal-oral mask may be positioned to cover both the patient's mouth and nose, and/or an oral mask may be positioned to cover the patient's mouth.

At block 204, the ventilation support system may be operated, e.g., by controller 104, to perform one or more therapeutic maneuvers (e.g., one or more of blocks 206-214) selected to cause targeted receptors (e.g., 114, 116) to initiate an autoregulation response by central nervous system 108. For example, at block 206, the system may incrementally increase and/or decrease CPAP applied to the patient by various amounts and at various intervals, e.g., to cause baroreceptors 116 to raise signals that cause central nervous system 108 to initiate one or more autoregulation responses. At blocks 208 and 210, the system may repeatedly activate and/or deactivate positive and negative airway pressure, respectively. This may also cause baroreceptors 116 to raise signals that cause central nervous system 108 to initiate one or more autoregulation responses.

At block 212, the system may incrementally alter a chemical composition of air supplied to the patient. For example the system may incrementally increase and/or decrease oxygen and/or nitrogen levels in the supplied air, e.g., to cause chemoreceptors 114 to raise signals that cause central nervous system 108 to initiate one or more autoregulation responses.

At block 214, the system may repeatedly/incrementally reposition one or more bodily aspects of the patient, or may instruct medical personnel to reposition one or more bodily aspects of the patient. For example the patient may be incrementally repositioned between supine and lateral positions. Additionally or alternatively, the patient's mandible or tongue may be repositioned (e.g., extended or retracted) incrementally. This may cause baroreceptors 116 to raise signals that cause central nervous system 108 to initiate one or more autoregulation responses. In some embodiments, repositioning of the patient may be confirmed by medical personnel, e.g., by the medical personnel making a record of the repositioning in a database, e.g., using a user interface associated with ventilation support system 100.

At block 216, the system may measure changes in vital signs that result from the autoregulation response(s) triggered at blocks 206-214. For example, vital sign sensor 124 may monitor the patient's blood pressure using a blood pressure cuff that is wrapped around the patient's arm and is likewise operably coupled to controller 104. In this manner, vital sign sensor 124 may provide measurements of change in blood pressure to controller 104. In some embodiments, controller 104 and/or vital sign sensor 124 may store these measurements, e.g., in memory 126 or in various databases. In some embodiments, in addition to or instead of having a built-in vital sign sensor, medical personnel make take various vital sign measurements at block 216 and enter those measurements into a database, e.g., using a user interface associated with ventilation support system 100.

At block 218, the system may analyze measured changes accumulated, e.g., by vital sign sensor and/or controller 104, to generate a cardiac, cardiopulmonary, and/or hemodynamic phenotype associated with the patient. For example, conditional and/or temporal correlation between the applied therapeutic maneuvers and the corresponding autoregulation responses (e.g., blood pressure changes effected by central nervous system 108) may be stored, e.g., in memory 126 or in another database. From this data, hemodynamic and other parameters that describe the patient's autoregulation responses may be extracted. In some embodiments, this data may be visualized, e.g., graphically, to provide medical personnel the data in a manner that is intuitive to understand.

At block 220, the patient may be matched or clustered with other patient's having similar phenotypes, e.g., having similar hemodynamic vectors. Clusters of patients having similar phenotypes may then be treated similarly, and results of that treatment may be studied, e.g., to improve medical diagnoses and medicinal strategies on a large scale.

At block 222, the system may operate the ventilation support system in accordance with the phenotype generated at block 218. For example, the hemodynamic parameters of the patient's phenotype may be implanted on ventilation support apparatus 102 and used to control it in an optimized manner. In instances where a patient's phenotype is generated using a ventilation support system that does not belong to the patient (e.g., it belongs to a hospital or other entity that performs sleep studies), the patient's phenotype (e.g., hemodynamic parameters) may be implanted on the patient's own ventilation support apparatus, such as their personal CPAP device. That way, the patient's personal CPAP device may be operated in an optimized manner.

The techniques described herein may have a variety of applications in addition to those already described above. For example, techniques disclosed herein may be used to screen patients who suffer from hypertension or are at risk to develop hypertension when physiological or environmental conditions are changed. The threshold of the patients' autoregulation systems may be analyzed to trigger increases of their blood pressures. As another example, patients may be screen who do not yet show symptoms of ventilation disorder but who only achieve a sufficient ventilation/perfusion balance using cardiac and respiratory compensation (e.g., CPAP device). As yet another example, compensation limits of patients may be screened to analyze boundaries in sympathetic autoregulation. Additionally or alternatively, autoregulation responses of patients may be induced to screen and monitor the patients' resulting compromised ventilation and/or cardiac stress. As yet more examples, techniques disclosed herein may be used to validate or reject applied ventilation support therapies, and/or to study and analyze a cardiac implication of such therapies, and/or to analyze cardiac relief of a simulated ventilation support therapy

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

1. A ventilation support system comprising:
a ventilation support apparatus;
a controller operably coupled with the ventilation support apparatus; and
memory storing instructions that are executable by the controller to:
operate the ventilation support apparatus to perform a therapeutic maneuver selected to cause one or more targeted receptors to initiate an autoregulation response in a patient connected to the ventilation support system;
determine, based on one or more signals received from a vital sign sensor, one or more changes in one or more vital signs caused by initiation of the autoregulation response in the patient;
generate a cardiac, cardiopulmonary, or hemodynamic phenotype of the patient based on the measured one or more changes; and
operate the ventilation support apparatus in accordance with the phenotype.

2. The ventilation support system of claim 1, wherein the one or more vital signs comprise blood pressure.

3. The ventilation support system of claim 2, wherein the controller is further configured to:
operate the ventilation support apparatus to alter an applied treatment parameter in a series of discrete steps; and
measure one or more changes in the blood pressure caused by each of the series of discrete steps.

4. The ventilation support system of claim 3, wherein the controller is further configured to operate the ventilation support apparatus to incrementally increase or decrease a continuous positive airway pressure.

5. The ventilation support system of claim 3, wherein the controller is further configured to operate the ventilation support apparatus to activate or deactivate positive or negative airway pressure.

6. The ventilation support system of claim 3, wherein the controller is further configured to operate the ventilation support apparatus to incrementally increase or decrease concentration of one or more components of air supplied to the patient.

7. The ventilation support system of claim 1, wherein the controller is further configured to:
provide output prompting medical personnel to reposition the patient between a plurality of bodily positions in a predetermined manner; and
measure one or more changes in blood pressure, or receive one or more indications of one or more changes in blood pressure, caused by the repositioning of the patient.

8. The ventilation support system of claim 1, wherein the one or more targeted receptors comprise one or more chemoreceptors, and wherein operating the ventilation support system to perform the therapeutic maneuver comprises operating the ventilation support system to alter a chemical composition of blood of the patient.

9. The ventilation support system of claim 7, wherein the one or more targeted receptors comprises one or more baroreceptors, and wherein operating the ventilation support system to perform the therapeutic maneuver comprises operating the ventilation support system to alter a vessel or airway pressure of the patient.

10. A method, comprising:
operating, by a controller communicatively coupled with a ventilation support apparatus connected to a patient, the ventilation support apparatus to perform a therapeutic maneuver selected to cause one or more targeted receptors of the patient to initiate an autoregulation response in the patient;
measuring, by the controller, one or more changes in blood pressure caused by initiation of the autoregulation response in the patient; and
generating, by the controller, a cardiac, cardiopulmonary, or hemodynamic phenotype of the patient based on the measured one or more changes.

11. The method of claim 10, wherein:
operating the ventilation support apparatus to perform the therapeutic maneuver comprises operating the ventilation support apparatus to alter an applied treatment parameter in a series of discrete steps; and
measuring the one or more changes in blood pressure comprises measuring one or more changes in the blood pressure caused by each of the series of discrete steps.

12. The method of claim 11, wherein operating the ventilation support apparatus to alter the applied treatment parameter in a series of discrete steps comprises operating the ventilation support apparatus to incrementally increase or decrease a continuous positive airway pressure.

13. The method of claim 11, wherein operating the ventilation support apparatus to alter the applied treatment parameter in a series of discrete steps comprises operating the ventilation support apparatus to activate or deactivate positive airway pressure.

14. The method of claim 11, wherein operating the ventilation support apparatus to alter the applied treatment parameter in a series of discrete steps comprises operating the ventilation support apparatus to activate or deactivate negative airway pressure.

15. The method of claim 11, wherein operating the ventilation support apparatus to alter the applied treatment parameter in a series of discrete steps comprises operating the ventilation support apparatus to incrementally increase or decrease a concentration of one or more components of air supplied to the patient.
